(19) United States
(12) Patent Application Publication
(10) Pub. No.: US 2014/0124448 A1
(43) Pub. Date: May 8, 2014

(54) IMMUNOAFFINITY SEPARATION MATERIALS COMPRISING ANTI-IgE ANTIBODY DERIVATIVES

(71) Applicants: Hans Huber, Vienna (AT); Christian Lupinek, Vienna (AT); Bernhard Maderegger, Vienna (AT); Gottfried Stegfellner, Asten (AT); Rudolf Valenta, Theresienfeld (AT); Josef Boeckmann, Wienerwald (AT); Friedrich Dorner, Vienna (AT); Wolfgang Schallenberger, Vienna (AT)

(72) Inventors: Hans Huber, Vienna (AT); Christian Lupinek, Vienna (AT); Bernhard Maderegger, Vienna (AT); Gottfried Stegfellner, Asten (AT); Rudolf Valenta, Theresienfeld (AT); Josef Boeckmann, Wienerwald (AT); Friedrich Dorner, Vienna (AT); Wolfgang Schallenberger, Vienna (AT)

(21) Appl. No.: 14/110,250

(22) PCT Filed: Apr. 13, 2012
(86) PCT No.: PCT/EP12/56809

§ 371 Date: Jan. 14, 2014

(30) Foreign Application Priority Data
Apr. 13, 2011 (EP) 11162331.0

(51) Int. Cl. A61M 1/34; C07K 16/42

(52) U.S. Cl. CPC A61M 1/3496; C07K 16/42
USPC 210/690; 530/391.1; 530/387.3

(57) ABSTRACT

The present invention provides an immunoaffinity separation material, comprising an antibody derivative having high specificity for soluble and cell bound IgE, an apheresis device comprising said material and its use for apheresis, specifically for plasmapheresis. It further provides a recombinant single chain antibody fragment with high specificity for soluble and cell bound IgE that is free of any tag sequences as well as the method for its production.

The present invention provides an immunoaffinity separation material, comprising an antibody derivative with high specificity for soluble and cell bound IgE and its use for plasmapheresis.

It further provides a single chain antibody fragment with high specificity for soluble and cell bound IgE that is free of any tag sequences as well as the method for its production.

### BACKGROUND OF THE INVENTION

Most allergic diseases are caused by IgE-mediated hypersensitivity reaction (type-1 hypersensitivity). IgE, a class of antibody/immunoglobulin normally present in the human plasma at minute concentrations, is produced by IgE-secreting plasma cells, which express the IgE-antibody on their surface at a certain stage of their maturation (differentiation). For reasons still not fully understood, allergic patients produce significantly increased amounts of IgE with binding specificity for ordinarily innocuous antigens (i.e. allergens) to which they are sensitive. These IgE circulate in the plasma and bind to IgE-specific receptors (FcεRI) on the surface of basophils in the circulation and mast cells along mucosal linings and underneath the skin.

During an allergic reaction, the inhaled or ingested allergens bind to IgE on mast cells and basophils, crosslink the IgE and aggregate the underlying receptors, thus triggering to release histamine, leukotrienes and other mediators of the symptomatic allergic response.

Furthermore binding of IgE to FcεRI-receptors on the surfaces of mast cells and basophils is mainly responsible for stable expression of such receptors on these effector cells.

Although therapeutic strategies have been suggested for targeting IgE, these therapeutic regimens provide systemic administration of anti-IgE antibodies, for example omalizumab, thus leading to unwanted side effects. Therefore, therapy using these approaches is limited to dosages which do not result in severe side effects, i.e. dosages that might not be sufficient for patients with high levels of IgEs showing symptoms of severe allergies.

EP434317A1 provides coupling methods for small specific binding agents having a molecular weight of not more than 25 kDa, especially Fv antibody fragments to affinity purification media.

WO95/31727 describes the immobilization of full length antibodies on sterile and pyrogen-free columns.

Tsumoto et al. (J. Immunol. Methods, 219(1998), 119-129) describe a method for the recovery of single chain fragments from inclusion bodies in the presence of oxidizing reagents.

Single-chain antibodies are refolded using different strategies to increase protein yields as described in Sinacola J. R. et al (Protein Expression and Purification, 2002, 26, 301-308).

US2009/0311750 describes the conversion of Fab molecules to scFv molecules.

There is still an unmet need to provide approaches for treatment of patients having high levels of IgE. There is also a need to provide systems for depletion of IgE specifically from patients with severe allergies, specifically with severe allergic asthma as well as to provide new antibody fragments which can be used for therapeutic purposes as well as for apheresis.

### DESCRIPTION OF THE INVENTION

This object is solved by the embodiments of the present invention.

The present invention provides an immunoaffinity separation material, comprising an antibody derivative immobilized on a solid material, said antibody derivative exhibiting specificity for soluble and cell bound IgE. The solid material may comprise any material known for affinity separation, for example porous solid phase carrier material. Preferably, the antibody derivative is covalently bound to said material. According to the invention the immunoaffinity material may be used for partial or complete removal of IgE from body fluid, specifically from blood, specifically from cell containing or cell-free blood fractions, more specifically from serum or plasma. An apheresis device comprising the separation material is also provided, specifically a plasmapheresis device, more specifically a device useful for performing extracorporeal apheresis or plasmapheresis.

IgE-specific extracorporeal immunoaffinity removes free IgE from blood of allergic patients, without forming immune complexes and substantial temporary increase of total IgE in the blood, therefore lacking such related serious clinical consequences. The present invention further provides the use of an antibody derivative exhibiting specificity for soluble and cell bound IgE, specifically of an scFv, more specifically of scFv12 comprising an amino acid sequence as shown in SEQ ID No. 1 (FIG. 2) in extracorporeal plasmapheresis treatment of a patient suffering from allergic disease. Specifically the antibody derivative is immobilized on an immunoaffinity separation material. Preferably, the antibody derivative as used is free of any tag sequences. The present invention further provides an antibody derivative for use in the treatment of a patient suffering from allergic diseases wherein the concentration of IgE in the organism is reduced by the steps of:

a) obtaining a sample of blood from said mammalian organism;
b) isolating the plasma from the cellular components from said blood sample;
c) contacting said isolated plasma with an immunoaffinity separation material as described above, whereby IgEs are retained on the immunoaffinity material;
d) reintroducing the cellular components isolated from step (b) and the purified plasma from step (c) to the patient,
e) and optionally repeating the steps c) and d) at least once Preferably, at least 80%, preferably at least 85%, more preferably at least 89% of the IgE antibodies may be removed from plasma.

According to a further embodiment of the invention, a recombinant single chain antibody fragment (scFv) exhibiting specificity for soluble and cell bound IgE which is free of any tag sequences is provided.

Said tag-free recombinant scFv may also be useful for preparing a medicament for the treatment of allergic diseases, specifically for the treatment of allergic asthma. Correct refolding of recombinant single chain antibodies expressed in host cells, specifically in bacterial cells is also provided by a method according to the invention. Therefore a method for obtaining a biologically active scFv exhibiting specificity for soluble and cell bound IgE from host cell inclusion bodies is provided, comprising the steps of:

a) solubilising host cell inclusion bodies with a solubilising agent, whereby the solubilising agent has a defined starting concentration,
b) reducing the disulfide bonds of the host cell expressed scFv by adding a reducing agent,
c) removing said reducing agent and concurrently reducing the concentration of the solubilising agent to an intermediate concentration of 6 to 60% of the starting concentration of said solubilising agent,
d) oxidizing the disulfide bonds of said scFv to produce biologically active scFv, whereby said oxidation step is performed at said intermediate concentration of the solubilising agent for at least 10 hours,
e) removing said solubilising agent,
f) isolating and optionally purifying biologically active scFv.

The intermediate concentration of said solubilising agent may be in the range of about 6 to 60% of the starting concentration, preferably about 15 to 40%, preferably about 30%. The solubilising agent may specifically be selected from guanidine hydrochloride (GuHCl) or urea, preferably it is GuHCl.

Specifically, the starting concentration of GuHCl is in the range of about 4 to 10 M, preferably about 5 to 7 M, most preferably about 6 M. The intermediate concentration of GuHCl is in the range of about 4 to 0.5 M, preferably it is in the range of about 3 to 1 M, most preferably it is about 2 M.

According to a specific embodiment, the oxidation step is performed for a sufficient time period to produce biologically active scFv, preferably it is performed for at least 24 hours.

It has been shown by the inventors that the oxidation step may by successfully performed also in the absence of any oxidizing agents.

Specifically, the amount of solubilising agent may be reduced step-wise by applying at least two, preferably at least three, preferably at least four, preferably at least five, preferably at least six dilution steps.

The reducing agent may be specifically selected from 2-mercaptoethanol (2-ME), cysteine and dithiothreitol (DTT), preferably it may be selected from 2-ME and DTT, preferably it is 2-ME.

Buffer exchange using membrane technology, preferably by dialysis, diafiltration, or dilution to remove the reducing agent or solubilising agent is provided according to a further embodiment.

According to a specific embodiment the reducing agent and/or the solubilising agent is removed by buffer exchange. Suitable technologies are e.g. dialysis, dia- or gel filtration or dilution. Preferably membrane technology such as dialysis or diafiltration is used.

### FIGURES

FIG. 1: Levels of total IgE and IgE specific to birch pollen, timothy grass pollen and to Dermatophagoides pteronyssinus are shown before, after the first and after the second run through the ScFv12-, the mAb12- and the control column.

FIG. 2: Amino acid sequence of scFv12

### DETAILED DESCRIPTION OF THE INVENTION

An immunoaffinity separation material is provided wherein an antibody derivative exhibiting specificity for soluble and cell bound IgE is immobilized on a solid material. Cell-bound IgE is immunoglobulin E which is bound to the FcεRI receptor on effector cells such as mast cells and basophils.

According to the present invention, the term “antibody derivative” may be any antibody fragment or derivative which comprises at least one antibody variable region and which has binding specificity for soluble and cell-bound IgE. Said derivatives may be, but are not limited to functional antibody fragments such as Fab, Fab2, scFv, Fv, or parts thereof, or other derivatives or combinations of the immunoglobulins such as nanobodies, diabodies, minibodies, single domains or Fab fragments, domains of the heavy and light chains of the variable region (such as Fd, VL, including Vlambda and Vkappa, VH, VHH) as well as mini-domains consisting of two beta-strands of an immunoglobulin domain connected by at least two structural loops.

Preferably the antibody derivative is monovalent and non-anaphylactic.

Preferably, the derivative is a single chain antibody fragment which selectively binds to soluble and/or cell-bound IgE.

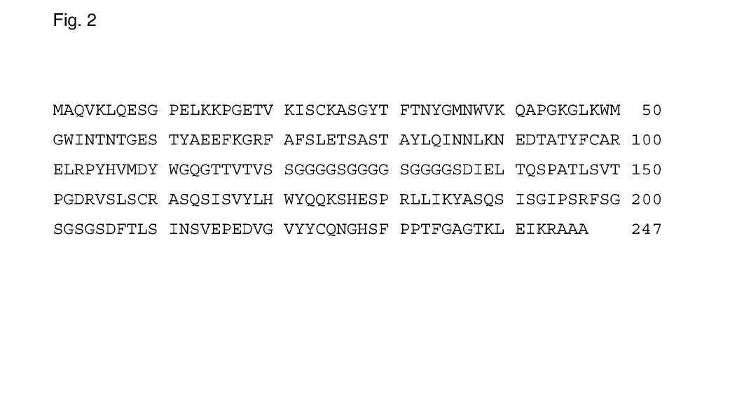

For example, it is a scFv12 having an amino acid sequence as shown in FIG. 2 (SEQ ID No. 1) or having at least 95%, specifically at least 98%, more specifically at least 99% sequence identity with SEQ ID No. 1 and as described in Lupinek et al. (2009). More preferably, it is the scFv12 as described in Lupinek et al. (2009) but free of any tag sequence. Said scFv12 preferably has a molecular mass of more than 25 kDa, i.e. about 26 kDa.

The immunoaffinity material used according to the invention can be any material known in the art which is suitable for affinity separation, like for example porous carrier materials, specifically porous solid phase carrier material. Specifically any conventional carrier material may be used, but is not limited to, agarose, sepharose, polysterene, controlled pore glass, dextrans, cellulose, synthetic polymers and co-polymers like hydrophilic polymers, porous amorphous silica.

The carrier materials may be particulate like beads or granules generally used in columns or in sheet form like membranes or filters which may be flat, pleated, hollow fibers or tubes.

Specifically, the material may be compressible, e.g. it is a soft or semi-rigid media, especially useful for apheresis

Preferably, the immunoaffinity material is sepharose, more specifically it is fast flow sepharose.

The antibody derivative of the invention can be coupled to the carrier material by known techniques either covalently or non-covalently.

The antibody derivative may be immobilized via a specific binding agent like a chemical group or a peptide group without significantly affecting the specific binding affinity.

Specifically, the antibody derivative is immobilized by covalent attachment onto the surface. Preferably, the antibody derivative is immobilized onto a periodate-oxidized carrier. Thereby, each of the dialdehyde groups of a periodate-oxidized nucleoside is coupling to lysine residues of the protein through Schiff bases, thereby cross-linking different protein molecules, forming a polymer.

It has been surprisingly shown by the inventors that scFv coupled on periodate-oxidized carrier does not significantly decrease its binding affinity to IgE. Therefore, an immunoaffinity material with covalently bound scFv having high affinity towards soluble and cell-bound IgEs is provided by the present invention. Unwanted leakage of the scFv from the carrier can thus be decreased or inhibited, which makes the material highly advantageous also for therapeutic purposes.

Specifically, the lysine residues involved in the coupling of scFv12 lacking the tag sequence are mainly found in the frame work regions but not in the CDR of scFv12 which may explain the unexpected maintained activity of coupled scFv12.

The method of coupling via a hydrophobic tail by non-covalent attachment described in EP434317 is not applicable in this regard because said method does not provide a stable matrix for the human use where leakage has to be reduced to the minimum.

Advantageously, the time for plasma or serum passing through the immunoaffinity material may be reduced compared to plasma passing through the same immunoaffinity material wherein a complete antibody is immobilized, still resulting in comparable reduction rates of IgE from said plasma. Preferably, using antibody derivatives, specifically scFvs, the pass through velocity is at least 10% increased, preferably at least 20% increased compared to using a complete antibody. This is specifically surprising as scFvs are binding to IgE via monovalent binding, whereas complete antibodies show divalent binding capacities.

The use of the inventive immunoaffinity material for reduction of IgE from body fluid is also provided by the present invention. According to a specific embodiment, the body fluid is serum or plasma.

An apheresis device comprising the immunoaffinity material according to the invention is also claimed, specifically a plasmapheresis device, which may be applicable for extracorporeal apheresis or plasmapheresis.

Apheresis is a method wherein the therapeutic effects are based on the extra-corporeal elimination of pathogenic proteins, protein-bound pathogenic substances, free pathogenic substances or pathogenic cells of the blood, in case of the present invention it is the removal of soluble and cell-bound IgE. If the pathogenic protein can only be eliminated from cell-free plasma, plasma previously is separated from the blood cells by means of a membrane plasma separator (plasma separation) or by means of a haemocentrifuge.

In the selective plasmapheresis method, which is the preferred method of the invention, IgEs are specifically removed from the separated plasma by adsorption, and it is possible to re-infuse the plasma without a substantial loss of volume after the removal has been effected. These selective methods have the advantage that it can be performed without a substitution solution.

In selective whole blood apheresis methods, the IgEs are specifically adsorbed directly from the non-pretreated blood without a previous plasma separation, whereby, in contrast to the plasma separation methods, both the plasma separation and the addition of a substitution solution can be omitted.

Therefore, the present invention also provides an antibody derivative exhibiting specificity for soluble and cell bound IgE for use in extracorporeal plasmapheresis treatment of an individual, specifically of a human patient, suffering from allergic disease, specifically, but not limited to allergic rhinoconjunctivitis, allergic asthma, urticaria and atopic dermatitis.

Allergic disease that can be treated by the present method is any disease caused by IgE-mediated hypersensitivity reaction. Specifically, but not limited to are severe allergic diseases caused by airborne allergens, more specifically seasonal allergies caused by allergens derived from grass, tree, or weed pollen and/or by perennial allergens from animal dander, moulds or house dust mites and cockroaches. Preferably for use in apheresis, the IgE specific antibody derivative, specifically a scFv, more specifically scFv12, free of any tag sequences is immobilized on an immunoaffinity separation material. More specifically, the invention provides the use of an IgE specific antibody derivative for reducing the concentration of IgE in a patient suffering from allergic disease comprising the steps of

a) obtaining a sample of blood from said patient organism;
b) isolating the plasma from the cellular components from said blood sample;
c) contacting said isolated plasma with an immunoaffinity separation material according to the invention, comprising the antibody derivative, whereby IgEs are retained on the immunoaffinity material;
d) reintroducing the cellular components isolated from step (b) and the purified plasma from step (c) to the patient,
e) optionally repeating steps c) and d) at least once.

Preferably, the patient is human.

It was surprisingly shown by the inventors that at least 80%, preferably at least 85%, more preferably at least 89% of the IgE antibodies can be removed from plasma by apheresis.

According to a further embodiment, a recombinant single chain antibody fragment (scFv) exhibiting specificity for soluble and cell bound IgE free of any tag sequences is provided which is advantageous for the treatment of allergic disease patients. Specifically, said scFv has a molecular weight of more than 25 kDa, more specifically it has a molecular weight of about 26 kDa, specifically about 26540 Da. More specifically, this scFv is of the same or similar amino acid sequence as scFv12 disclosed in Lupinek et al. (2009) but lacking any tag sequences thus making the scFv molecule more advantageous in view of therapeutic application. Such tags are fused to the N-terminus or the C-terminus of the scFv and are usually used for simplified analytical detection or affinity purification (e.g. E-tag, 6-His-tag, S-tag, glutathione tag, TEV tag, etc.). However, such tags do not have any therapeutic advantage. Moreover, in the case of leaching of the tagged scFv from the immuno-affinity separation material, and its subsequent reintroduction into the patient, the tag or the tagged scFv may have a disadvantageous side effect in the patient organism. In addition, it was surprisingly found by the inventors that the presence of a tag may have an undesired influence on the biological activity of the scFv (i.e. reduced IgE binding affinity due to the tag). The use of a tag-free scFv is therefore highly preferable.

According to the invention, the term “biologically active” means that the antibody derivative, specifically the scFv exhibits specific binding to soluble and cell-bound IgE.

The antibody derivatives can be produced in cell culture. For expressing scFv, any host cell system can be used known for the expression of antibodies or antibody derivatives like scFv. Thus this can be any applicable animal, plant, bacterial, filamentous fungal or yeast host cell system. Specifically and preferably, the host cells are bacterial cells like Escherichia coli or Pseudomonas fluorescens, wherein these scFvs are produced as cytoplasmic inclusion bodies (refractile bodies) which have to be correctly refolded in vitro thereafter. Methods for refolding of scFv or other antibody derivatives like nanobodies or single domain antibody fragments have been reported in prior art. Such methods involve diluting solubilised proteins with a refolding buffer, however these methods are time consuming and involve laborious steps. Alternatively, the host cells are yeast cells like Pichia pastoris, Hansenula polymorpha, Saccharomyces cerevisiae or any other yeast cells known in the art which are capable of extracellular secretion of the antibody derivatives into the culture medium. Examples of antibody derivatives that can be secreted with yeast cells include scFv, Fab and nanobodies.

The inventors have successfully established a method for obtaining a biologically active scFv exhibiting specificity for soluble and cell bound IgE from host cell inclusion bodies, comprising the steps of:
a. solubilising said inclusion bodies with a solubilising agent, whereby the solubilising agent has a starting concentration, specifically of 4 to 10 M,
b. reducing the disulfide bonds of said scFv by adding a reducing agent,
c. removing said reducing agent and concurrently reducing the concentration of the solubilising agent to an intermediate concentration of 6 to 60% of the starting concentration of said solubilising agent,
d. oxidizing the disulfide bonds of said scFv to produce biologically active scFv, whereby said oxidation step is performed at said intermediate concentration of the solubilising agent for at least 10 hours,
e. removing the residual solubilising agent,
f. isolating and optionally purifying biologically active scFv.

Specifically the method is used for refolding and thus obtaining biologically active scFv12.

Solubilising the inclusion bodies can be performed for example under following specific conditions: The concentration of inclusion bodies for performing the step of solubilising is between 0.01 and 200 g/L specifically between 0.0.5 and-100 g/L, more specifically from 0.1 to 50 g/L. The solubilising agent may be guanidine hydrochloride (GuHCl) or urea, preferably it is GuHCl.

The term “starting concentration” means a suitably high concentration of solubilising agent enabling the solubilisation of the inclusion bodies. For example, if GuHCl is used, the starting concentration is from 4 to 10 M, preferably 5 to 7 M, most preferably about 6 M.

Any standard buffer system can be selected known in the art, for example it can be Tris buffer, specifically at a concentration from about 10 to 100 mM and/or borate at a concentration of about 100 mM. Optionally salts can be added such as for example NaCl, specifically about 200 mM of NaCl. The optimum pH conditions are neutral to alkaline and shall not be acidic, specifically the pH is in the range from 7 to 14, preferably from 7 to 12, most preferably from 8 to 9. The time for performing the solubilising step may range between 0 and 200 h, preferably between 0.1 and 72 h, most preferably between 1 and 36 h.

Reducing the disulfide bonds of said scFv is performed by adding a reducing agent like for example 2-mercaptoethanol (2-ME), cysteine, dithiothreitol (DTT). Preferably the reducing agent is 2-ME or DTT, most preferably it is 2-ME. When 2-ME is used, the concentration of 2-ME is from about 0.1 to 100 mM, preferably it is from about 1 to 20 mM, most preferably it is from about 5 to 15 mM.

The time for performing the reducing step can be determined by the skilled person, for example it is in the range from 0 to 200 h, preferably from 0.1 to 72 h most preferably from 1 to 36 h.

The method for removing said reducing agent and concurrently reducing the concentration of the solubilising agent to an intermediate concentration of 10 to 60% of the starting concentration of said solubilising agent is for instance performed by buffer exchange using conventional membrane technology like for example dialysis, diafiltration (hollow fibre, cassettes) or dilution. Preferably the residual concentration of the reducing agent is below 0.5 mM, preferably below 0.1 mM at the stage of the intermediate concentration of the solubilising agent.

Membrane technologies are processes which allow the separation of the different components of a fluid based on their size. The right choice of membrane cut-off can for example enable the removing of the reducing agent. The membrane cut off of the membrane technology is from 1 to 50 kDa, preferably from 5 to 25 kDa, most preferably about 10 kDa. The dilution factor may be from 0.1 to 100000, preferably from 5 to 1000, most preferably from 10 to 100. The “dilution factor” is defined as the ratio between the starting concentration of the buffer and the final target concentration of said buffer, whereby in this context, the “buffer” is the solubilising agent or the reducing agent, etc.

Specifically, the intermediate concentration of the solubilising agent is from 6 to 60% of the starting concentration, preferably from 15 to 40%, preferably about 30%.

In case the solubilising agent is GuHCl, the intermediate concentration is from 4 to 0.5 M, preferably from 3 to 1 M, most preferably 2 M.

It is essential that after solubilisation of the inclusion body and after the reduction of the disulfide bonds of the antibody derivative, said disulfide bonds are oxidised in order to facilitate the refolding of the protein and the formation of its soluble, stable and biologically active three-dimensional structure (tertiary structure). It was surprisingly shown by the inventors that for obtaining the highest refolding yield (i.e. the highest percentage of soluble, stable and biologically active scFv), two factors are essential, (1) the absence of reducing agent and (2) the presence of an intermediate concentration of solubilising agent. It is further essential that for obtaining the highest refolding yield, the oxidizing conditions at the stage of intermediate concentration of solubilising agent have to be maintained for a certain time period which is 10-200 h, preferably 10-72 h and most preferably 10-36 h.

The disulfide bonds of said scFv are oxidized to produce biologically active scFv, said oxidation step is performed at said intermediate concentration of the solubilising agent for instance for at least 10 hours, preferably for at least 24 hours. The oxidising procedure can be performed by adding an oxidation agent which may be, but is not limited to cystine, a dimer of glutathione (GSSG) or metal ions (Cu⁺⁺), specifically cystine may be selected. Even more specifically, the concentration of cystine is from 0.01 to 10 mM, preferably from 0.1 to 5 mM, most preferably from 0.5 to 1 mM. Surprisingly, it has been shown by the inventors that oxidation of the disulfide bonds of the antibody derivative takes place even in the absence of an oxidizing agent (i.e. without separate addition of an oxidizing agent). The benefits of avoiding the addition of oxidizing agents are decreased material costs and simplified purification, Therefore, most preferably, the oxidation step of scFv is conducted in the absence of any additionally added oxidation agent.

During and after performing oxidation of scFv, the amount of solubilising agent is removed by the application of one or more buffer exchange steps. The removal of the solubilising agent (i.e. buffer exchange) can be done again by using membrane technology like dialysis, diafiltration (hollow fibre, cassettes) or dilution. The specific dilution factor may be between 0.1 and 100000, preferably between 5 and 1000, most preferably between 10 and 100. Specifically, the membrane cut off may be selected according to the size of the scFv molecule, specifically it is in the range from 1 to 50 kDa, preferably from 5 to 25 kDa, most preferably at a membrane cut off of about 10 kDa.

Optionally, stabilizing agents can be added during the oxidation process, for example L-arginine, for instance in an amount of 0.4 to 1 M.

As a final step of the method, isolating and optionally purifying the biologically active scFv is performed.

Any contaminating agents or impurities can again be removed by buffer exchange using membrane technology or dilution. According to a specific embodiment, the optimum final buffer preferably is column coupling buffer, e.g. borate. The specific membrane cut off is again from 1 to 50 kDa, preferably from 5 to 25 kDa, most preferably about 10 kDa. The specific dilution factor may be between 0.1 and 100000, preferably between 5 and 1000, most preferably between 10 and 100. The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### EXAMPLES

### Example 1

### Manufacturing Process for scFv Exhibiting Specificity for Soluble and Cell Bound IgE (Anti-IgE-scFv)

Anti-IgE-scFv is manufactured by applying the following manufacturing process: The DNA sequence coding for anti-IgE-scFv was cloned into an expression vector (e.g. pET28b) and the resulting expression plasmid was transformed into competent cells of Escherichia coli BL21(DE3). Plasmid-carrying clones were selected and cultured in a culture medium containing glucose, mineral salts and trace elements by using a bioreactor (fermenter). A high cell density fed-batch cultivation procedure was applied by using a glucose-limited exponential feeding procedure. At an optical density of OD600=40-50, the recombinant expression of anti-IgE-scFv was initiated (induced) by the bolus addition of IPTG. After five hours of induced phase, the fermentation was terminated and the biomass (bacterial cells) containing the anti-IgE scFv was harvested by centrifugation. The bacterial cells were homogenized by application of a high-pressure homogenizer. Inclusion bodies which contain the anti-IgE-scFv in their inactive, insoluble and aggregated form, were obtained by centrifugation and washing of the homogenization suspension as described in Example 2. The inactive anti-IgE-scFv from the inclusion bodies was transformed into its active conformation by a refolding (renaturation) process as described in detail in Example 3. The refolding procedure consists of solubilization of the inclusion bodies with guanidine hydrochloride and reducing disulfide bonds with a reducing agent, followed by a step-wise dilution in order to renaturate the anti-IgE-scFv into its native and active conformation. After refolding, the protein was purified by the application of several chromatographic principles (hydrophobic interaction, ion exchange, size exclusion). For obtaining the final buffer conditions, an ultra-diafiltration step was applied in order to generate the optimum coupling conditions (final borate buffer). The bulk product of anti-IgE-scFv was then sterile-filtered by using a disposable sterile filter and then aseptically aliquoted into sterile product containers. The product was then frozen and stored at the temperature below −20° C.

### Example 2

### Preparation of Inclusion Bodies

### 1. High Pressure Homogenization.

Frozen biomass (−20° C.) was resuspended in a resuspension buffer containing 20 mM TrisHCl and 1 mM EDTA at pH=8.0 (250 g biomass per litre buffer). Cell suspension was thawed at room temperature under mechanical agitation for 30 min. Remaining frozen biomass was resuspended using an agitation device (Ultra-Turrax) for 1 min at about 15,000 rpm. Thawed cell suspension was subjected to a high pressure homogenizer (GEA, Panda 1K-NS1001L) for three passages at 750 bar. During homogenization, the homogenate was cooled down to 15° C. by using a heat exchanger. The crude cell homogenate was subjected to a centrifugation step at 7,000 rpm (5,500 g) and 4° C. for 60 min. The inclusion body pellets were collected and subjected to the following wash procedure.

2. Inclusion Body Washing with Triton:

Inclusion bodies containing anti-IgE-scFv were resuspended at a concentration of 60-75 g per liter of Triton washing buffer (20 mM Tris, 1 mM EDTA, 1% Triton X-100, pH 8.0) using an Ultra-Turrax for 1 min at about 15.000 rpm. Afterwards, the suspension was stirred at room temperature for 30 min and afterwards centrifuged at 7.000 rpm (5,500 g) and 4° C. for 60 min. This procedure was performed three times.

3. Inclusion Body Washing with Ethanol:

Inclusion bodies containing anti-IgE-scFv were resuspended at a concentration of 100-150 g per liter of ethanol washing buffer (20 mM Tris, 1 mM EDTA, 50% ethanol, pH 8.0) by using an Ultra-Turrax for 1 min at about 15,000 rpm. Afterwards the suspension was centrifuged. This procedure was performed twice.

### Example 3

### Refolding Process for Anti-IgE-scFv by Applying Step-Wise Dialysis

### 1. Solubilization of Inclusion Bodies and Reduction of Disulfide Bonds:

Washed inclusion bodies were solubilized in a solubilization buffer (0.5-1.0 g inclusion bodies per L). The solubilization buffer contained 6 M guanidine-hydrochloride (GuHCl), 50 mM Tris-HCl, 200 mM NaCl and 1 mM EDTA at pH=8.0. The inclusion bodies were solubilized by agitation at room temperature for the time period of at least 30 min. After solubilization, the reducing agent 2-mercaptoethanol was added (10 mM final concentration), thereby reducing the disulfide bonds of the anti-IgE-scFv. This solution was agitated at room temperature for at least 30 min.

### 2. Step-Wise Dialysis (Refolding Step):

The 2-mercaptoethanol was removed by dialysis (membrane cut-off 10 kDa) against the same solubilization buffer as described above (at 4° C. for 15 h).

GuHCl was removed by applying a number of serial dialysis steps (dilution factor 40-60) against the solubilization buffer as described above but containing de-creasing concentrations of GuHCl (e.g. 4, 3, 2, 1, 0.5 and 0 M GuHCl). Six dialysis steps were performed for removal of GuHCl. Each dialysis step was performed at 4° C. for 8-15 h. The anti-IgE-scFv is refolded into its soluble conformation as a consequence of the removal of GuHCl, in combination with oxidizing conditions for a certain time period.

At the dialysis steps of 1 and 0.5 M GuHCl, cystine was added at the concentration of 0.5-1 mM in order to oxidize the disulfide bridges of the anti-IgE-scFv. Furthermore, at the dialysis steps of 1 and 0.5 M GuHCl, arginine hydrochloride can be added (0.4 M) in order to increase the refolding yield.

### 3. Concentration Adjustment and Dialysis Against Coupling-Buffer

The solution containing the refolded protein was concentrated by a factor 10 by applying an ultrafiltration step (10 kDa membrane, Vivaspin 15, Sartorius) Afterwards, two consecutive dialysis steps (dilution factor=100) were performed against the coupling buffer containing 100 mM boric acid and 200 mM NaCl (pH=9.0-9.5). The dialysis steps were carried out at 4° C. for 15-24 h.

### Example 4

### Refolding Process for Anti-IgE-scFv by Applying Dilution and Dialysis

### 1. Solubilisation of Inclusion Bodies and Reduction of Disulfide Bonds:

Washed inclusion bodies were solubilized at a concentration of 16.7 g per L (solubilization buffer as described in 4) at 4° C. for 48 h. Afterwards, the solubilised anti-IgE-scFv was reduced by addition of 3.33 mM 2-mercaptoethanol (>60 min at room temperature).

### 2. Direct Dilution to Intermediate GuHCl Concentration:

Solubilized anti-IgE-scFv (15 mL) was directly diluted into 485 mL of a buffer containing 2 M GuHCl (=intermediate concentration of solubilising agent), 50 mM Tris-HCl, 200 mM NaCl, 1 mM EDTA and 0.4 M arginine hydrochloride (pH=8.0). Dilution factor was 33 and addition rate was about 0.2 mL/min. The diluted solution was incubated at 4° C. for 15 h.

### 3. GuHCl Removal:

GuHCl was removed in a step-wise mode by the performance of 2-3 consecutive dialysis steps (dilution factor 40-60) against a buffer containing 50 mM Tris-HCl, 200 mM NaCl and 1 mM EDTA (pH=8.0) or a buffer containing 100 mM boric acid and 200 M NaCl (pH=9.0-9.5) and in addition, decreasing concentrations of GuHCl in each step (e.g. 1, 0.5 and 0 M or 0.75 and 0 M). Each dialysis was performed at 4° C. for 8-15 h

At the dialysis steps of 1 and 0.5 M GuHCl, cystine was added at the concentration of 0.5-1 mM in order to oxidize the disulfide bridges of the anti-IgE-scFv. Furthermore, at the dialysis steps of 1 and 0.5 M GuHCl, arginine hydrochloride can be added (0.4 M) in order to increase the refolding yield.

As an alternative mode for GuHCl removal, GuHCl can be removed by one singular buffer exchange step (e.g. dialysis or diafiltration) against a buffer containing 50 mM Tris-HCl, 200 mM NaCl, 1 mM EDTA and 0.5-1 mM cystine (pH=8.0) or a buffer containing 100 mM boric acid, 200 M NaCl and 0.5-1 mM cystine (pH=9.0-9.5).

### Example 5

### Refolding Process for Anti-IgE-scFv by Applying Dilution and Dialysis (without Oxidation Agent)

The procedure was carried out as described in Example 4, but without using cystine as oxidation agent. Similar results in view of correct refolding of the scFv were received using this method (data not shown) compared to refolding in the presence of an oxidising agent.

### Example 6

### Purification of mAb12

Monoclonal antibody 12 was purified from culture supernatant of hybridoma-cells that were grown in serum-free medium by affinity chromatography using protein G-sepharose. Binding of purified mAb12 to human IgE was confirmed by ELISA.

### Example 7

### Development of a ScFv-Based Immunoadsorber Introduction and Experimental Design

The capacity of a tag-free single-chain variable fragment (scFv) derived from an anti-human IgE antibody (mAb12) to be used for the construction of an immunoadsorber for the selective depletion of IgE from human serum/plasma was investigated.

To compare the capacity of the same ScFv12 in Lupinek et al. (Lupinek et al. Trimolecular complex formation of IgE, Fc(epsilon)RI, and a recombinant nonanaphylactic single-chain antibody fragment with high affinity for IgE. J Immunol 2009; 182:4817-29.), but free of a tag and mAb12 (Laffer et al. A high-affinity mono-clonal anti-IgE antibody for depletion of IgE and IgE-bearing cells. Allergy 2008; 63:695-702.) to deplete IgE from serum/plasma, two columns were generated that are comparable with respect to the same number of IgE-binding paratopes immobilised to the sepharose matrix. For immobilisation, 5 mg of ScFv12 (Mw=28 kDa) and a corresponding amount of mAb12 (Mw=150 kDa), i.e., 12.5 mg, were used for coupling. When applying serum/plasma to the column, the sample is diluted due to the buffer volume contained in the sepharose bed. In order to estimate the dilution factor and to compare results, both the ScFv12- and the mAb12-columns were designed to have the same bed volume (i.e., approximately 5 ml each) and additionally, a third column was generated without any protein being immobilised. When calculating the capacity of the columns, 5 mg of ScFv12, like 12.5 mg of mAb12, can bind 30 mg of IgE, 100% saturation provided. 100% saturation cannot be achieved for different reasons like activity of the immobilised proteins being less than 100%, loss of activity during the coupling procedure, inaccessibility of the paratope after coupling, low IgE-concentration in the sample, etc. Therefore, the actual amount of IgE bound to the adsorber will be markedly below this theoretical value. The following experiments were performed to experimentally approach the capacity of the columns in order to calculate the volume of an immunoadsorber required to deplete IgE from a defined plasma-volume containing a defined concentration of IgE and to generate baseline values for the optimisation process of the coupling and the depletion procedure.

### Methods

Description of mAb12 and ScFv12

Data characterising both mAb12 and ScFv12, including the DNA- and protein-sequence of ScFv12 have been published before (Lupinek et al. 2009 and Laffer et al., 2008, see above)

Coupling of ScFv12 and mAb12 to Sepharose

Sepharose 4 Fast Flow from GE-Healthcare (Buckinghamshire, UK) was used. To activate the sepharose, 15 ml of the resin were equilibrated with 0.4% (w/v) NaIO₄ and incubated with 30 ml of 0.4% (w/v) NaIO₄ for 3 hours at room temperature with gentle shaking. Following activation, the sepharose was washed 5 times with Milli-Q water and equilibrated with borate-buffer (100 mM H₃BO₃, 200 mM NaCl, pH 9-9.5). Three 5 ml aliquots of the resin were transferred to 15 ml tubes. Five mg of ScFv12 or 12.5 mg of mAb12, both dissolved in borate buffer were added, volumes were adjusted with borate buffer to a final volume of 12 ml. For the empty column, only borate buffer was added to a final volume of 12 ml. Proteins were allowed to bind by incubation at 4° C. overnight on a shaker.

For deactivation of the sepharose after 20 hours of coupling, the resins were transferred to polypropylene columns, flow-throughs were collected and pooled with flow-throughs from the consecutive washing step to determine coupling efficiency. After washing with borate buffer the sepharose was equilibrated with 0.3% NaBH₄ and incubated for 12-15 minutes at room temperature. After blocking, the columns were thoroughly rinsed with PBS and stored at 4° C.

### Preparation of Plasma Samples for Depletion

From a patient with highly elevated total IgE levels (please refer to results), several plasma samples that had been diluted 1:2 in PBS were pooled to obtain a total volume of 140 ml. Protein precipitates were removed by centrifugation and filtration (Steritop Express Plus Membrane, Millipore, Darmstadt, Germany).

Depletion of IgE from Plasma Samples

After equilibration of the columns with PBS, 40 ml of plasma were applied on each column. Flow-throughs were collected and applied a second time. Aliquots were obtained from every sample for analysis. The time required for the 40 ml-sample to pass through the ScFv12-column was 15 minutes compared to 22 minutes for the mAb12-column and 27 minutes for the column with no protein immobilised.

The columns were washed with approximately 20 ml PBS, bound IgE was eluted with 9 ml of 5 M MgCl₂. The first 2 ml of the elution fraction were discarded, 7 ml were collected. The solvent of the elution fractions was changed to PBS and proteins were concentrated using Amicon Ultra-15 tubes (Millipore) with a cut off of 10 kDa. Columns were washed with PBS, equilibrated with water containing 0.02% NaN₃ and stored at 4° C.

### Analysis of Flow-Throughs and Eluted Proteins

In the plasma samples obtained before and after every run through the columns, total IgE and IgE specific to birch pollen, timothy grass pollen and Dermatophagoides pteronyssinus were determined by ImmunoCAP (Phadia, Uppsala, Sweden).

Protein concentrations in flow-throughs and wash fractions collected after coupling and in elution fractions were determined by Micro BCA protein assay kit (Pierce, Rockford, Ill., USA). Eluted samples were also analysed by SDS-PAGE.

### Results

### Coupling Efficiencies

In the flow-throughs and wash fractions obtained after coupling, 1.2 mg of mAb12 and 0.8 mg of ScFv12 were measured, corresponding to 10% of mAb12 and 16% of ScFv12 added for immobilisation. To account for loss of protein during concentration of the samples prior to measuring protein concentrations, the amounts of uncoupled mAb12 and ScFv12 were estimated to be higher, i.e., 15% for mAb12 and 20% for ScFv12.

Therefore, in the present experiment coupling efficiencies were approached to be 85% for mAb12 and 80% for ScFv12, corresponding to 10.6 mg mAb12 and 4 mg of ScFv12 immobilised to the sepharose matrix.

### Reduction of IgE Concentrations in Plasma Samples

The total IgE level in the plasma sample applied on the columns was determined to be 2129 kU/I, IgE specific to birch pollen was 14.9 kUA/I, to timothy grass pollen 59.3 kUA/I and to Dermatophagoides pteronyssinus 52 kUA/I.

After the first run through the ScFv12-column, IgE-levels were reduced by more than 80%, after the second run by almost 90% in total, opposed to almost complete depletion of IgE already after the first run through the mAb12-column and only less than 10% reduction of total IgE and between 3 and 15% reduction of specific IgE-levels after passage through the control column. In the latter, virtually no differences in IgE-levels were detected between the first and the second run.

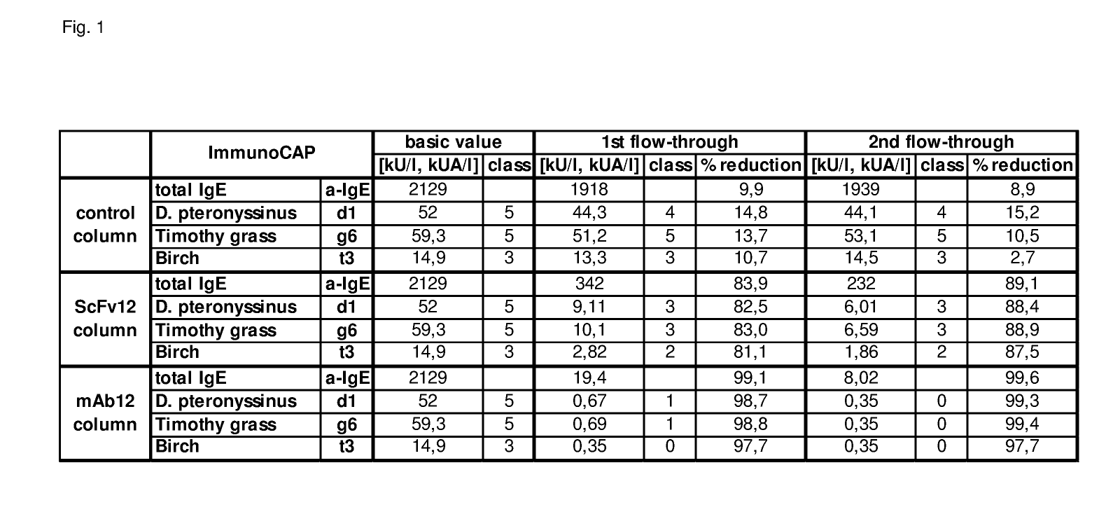

All results are shown in FIG. 1.

### Calculated Amounts of Depleted IgE

With 1 international unit of IgE corresponding to 2.4 ng of IgE (Bazaral M, Hamburger RN. Standardization and stability of immunoglobulin E (IgE). J Allergy Clin Immunol 1972; 49:189-91), 40 ml of plasma with a total IgE-level of 2129 kU/I contain approximately 200 μg of IgE.

According to ImmunoCAP results, in total 18 μg of IgE were depleted by 2 runs through the control column, 180 μg of IgE by the ScFv12-column and 200 μg by the mAb12-column.

These results are within the range of the amounts of IgE measured in the elution fractions which were 220 μg for the ScFv12-column and 270 μg for the mAb12-column. Elution fractions of the three columns were also analysed by SDS-PAGE (data not shown), revealing slight non-specific interaction of plasma proteins with the sepharose-matrix. In previous depletion experiments with smaller adsorber-volumes, beside a strong IgE-signal, IgG could also be detected in elution fractions by ELISA (data not shown). Therefore, for the calculation of the total amounts of IgE in the elution fractions of the ScFv12- and the mAb12-columns, the respective protein concentrations were reduced by concentrations measured in the elution fraction from the control column.

1. An immunoaffinity separation material, comprising an antibody derivative immobilized on a solid material, wherein said antibody derivative exhibits specificity for soluble and cell bound IgE.

2. The immunoaffinity material according to claim 1, wherein said material comprises porous solid phase carrier material.

3. The immunoaffinity material according to claim 1, wherein the antibody derivative is covalently bound to the solid material.

4. A method for removing IgE from body fluid, preferably from serum or plasma, comprising the step of contacting the body fluid with the immunoaffinity material of claim 1.

5-14. (canceled)

15. A method for obtaining a biologically active scFv exhibiting specificity for soluble and cell bound IgE from host cell inclusion bodies, comprising the steps of:
a) solubilising said inclusion bodies with a solubilising agent, whereby the solubilising agent has a starting concentration,
b) reducing the disulfide bonds of said scFv by adding a reducing agent,
c) removing said reducing agent and concurrently reducing the concentration of the solubilising agent to an intermediate concentration of 6 to 60% of the starting concentration of said solubilising agent,
d) oxidizing the disulfide bonds of said scFv to produce biologically active scFv, whereby said oxidation step is performed at said intermediate concentration of the solubilising agent for at least 10 hours,
e) removing said solubilising agent,
f) isolating and optionally purifying biologically active scFv.

16. The method according to claim 15, wherein the scFv is scFv12.

17. The method according to claim 15, wherein the intermediate concentration is from 15% to 40%, and is preferably about 30%.

18. The method according to claim 15, wherein the solubilising agent is guanidine hydrochloride (GuHCl) or urea, and preferably is GuHCl.

19. The method according to claim 18, wherein the starting concentration of GuHCl is from 4 M to 10 M, preferably 5 M to 7 M, and more preferably is about 6 M.

20. The method according to claim 15, wherein the solubilising agent is GuHCl and the intermediate concentration of the GuHCl is from 4 M to 0.5 M, preferably 3 M to 1 M, and most preferably is 2 M.

21. The method according to claim 15, wherein the step of oxidizing the disulfide bonds of said scFv is performed for least 24 hours.

22. The method according to claim 15, wherein the oxidizing step is performed in the absence of oxidizing agents.

23. The method according to claim 15, wherein the reducing agent is selected from the group consisting of 2-mercaptoethanol (2-ME), cysteine and dithiothreitol (DTT), preferably is selected from the group consisting of 2-ME and DTT, and most preferably is 2-ME.

24. The method according to claim 15, wherein said reducing agent or solubilising agent is removed by buffer exchange using membrane technology, preferably by dialysis, diafiltration or dilution.

25. A method for treating a patient using extracorporeal plasmapharesis with an antibody derivative exhibiting specificity for soluble and cell bound IgE, wherein the patient is suffering from allergic disease, preferably a disease selected from the group consisting of allergic rhinoconjunctivitis, allergic asthma, urticaria and atopic dermatitis.

26. The method according to claim 25, wherein the antibody derivative is immobilized on an immunoaffinity separation material.

27. The method according to claim 25, wherein the antibody derivative is a single chain antibody fragment (scFv).

28. The method according to claim 27, wherein the antibody derivative has the sequence of SEQ ID NO:1.

29. The method according to claim 25, wherein the antibody derivative is free of any tag sequences.

30. The method according to claim 25, wherein the concentration of IgE in a patient organism is reduced by:
a) obtaining a sample of blood from said mammalian organism;
b) isolating the plasma from the cellular components from said blood sample;
c) contacting said isolated plasma with an immunoaffinity separation material, whereby IgEs are retained on the immunoaffinity material;
d) reintroducing the cellular components isolated from step b) and the purified plasma from step c) to the patient,
e) and optionally repeating steps c) and d) at least once.


SEQUENCE LISTING (1 sequence)

SEQ ID NO: 1 - protein, 247 aa, Artificial Sequence
  single-chain variable fragment
MAQVKLQESGPELKKPGETVKISCKASGYTFTNYGMNWVKQAPGKGLKWMGWINTNTGESTYAEEFKGRFAFSLETSASTAYLQINNLKNEDTATYFCARELRPYHVMDYWGQGTTVTVSSGGGGSGGGGSGGGGSDIELTQSPATLSVTPGDRVSLSCRASQSISVYLHWYQQKSHESPRLLIKYASQSISGIPSRFSGSGSGSDFTLSINSVEPEDVGVYYCQNGHSFPPTFGAGTKLEIKRAAA